(19) United States
(12) Patent Application Publication
(10) Pub. No.: US 2024/0122668 A1
(43) Pub. Date: Apr. 18, 2024

(54) MEDICAL INSTRUMENT FOR TREATING A BODY WITH A PRESSURE RELIEF DEVICE

(71) Applicant: KARL STORZ SE & Co. KG, Tuttlingen (DE)

(72) Inventors: Beat KRATTIGER, Tuttlingen (DE); Simon LEHMANN, Tuttlingen (DE)

(21) Appl. No.: 18/379,432

(22) Filed: Oct. 12, 2023

(30) Foreign Application Priority Data
Oct. 14, 2022 (DE) 10 2022 126 989.2

(51) Int. Cl. A61B 90/00; A61B 17/22

(52) U.S. Cl. CPC A61B 90/03; A61B 17/22012; A61B 2017/00862; A61B 2017/22025; A61B 2017/22079

(57) ABSTRACT

The invention relates to a medical instrument for treating a body, wherein the medical instrument has a carrier unit having an outside to an environment and at least one first interior space within the carrier unit, and an overpressure can be applied to the interior space, wherein the medical instrument has a pressure relief device having at least one elastic sealing element, wherein the at least one elastic sealing element is arranged in a first recess in and/or on the outside of the carrier unit and the at least one interior space is connected to the first recess by means of at least one pressure relief channel, such that, when the overpressure is applied to the at least one interior space, the overpressure can be discharged into the environment by means of the first recess being freed by the at least one elastic sealing element.

### CROSS-REFERENCE TO RELATED APPLICATIONS

This application claims the benefit under 35 U.S.C. 119(a) to German Patent Application No. 10 2022 126 989.2, filed 14 Oct. 2022, the disclosure of which is incorporated herein by reference in its entirety.

### BACKGROUND

The invention relates to a medical instrument for treating a body, wherein the medical instrument has a carrier unit having an outside to an environment and at least one first interior space within the carrier unit, and an overpressure can be applied to the interior space.

In medical instruments, a pressure medium is often used in an interior space of the instrument, with a hollow tool or a hollow probe being at least partially inserted into the patient. There is a potential risk to the patient in the event of an undesired escape of pressure medium, such as compressed air or a heat fluid, from the interior space of the instrument into the hollow tool or the hollow probe. In the event of an instrument fault, compressed air, for example, may accidentally enter the patient's body, infecting the patient with germs and/or mechanically injuring the patient through the overpressure.

For example, lithotripsy is a known method of crushing stones in the body which form, for example, through condensation and/or crystallization of salts and proteins as so-called concretions in body organs, for example in the bladder or kidneys. If these stones are too large to be passed naturally and they cause discomfort, they have to be crushed with a lithotripter so that the crushed stones can be removed by natural excretion and/or by means of a suction/irrigation pump. In ballistic lithotripters, a projectile is accelerated within an acceleration tube, and the kinetic energy of the projectile is transferred via an elastic shock to the proximal end of a probe and onward to the distal end of the latter for fragmenting a stone in the body. Irrespective of the way in which the kinetic energy is generated, a compression of a gas and/or air volume always takes place in ballistic lithotripters due to the movement of the projectile in the acceleration tube, and a corresponding overpressure occurs, which can penetrate as far as a patient in the event of a fault in the pressurized region of the lithotripter. The risk to the patient is further increased in the case of pneumatic lithotripters, in which compressed air is used specifically to accelerate the projectile within the acceleration tube. Especially in the case of pneumatic lithotripters, there is an increased danger of compressed air being able to enter the patient's body, for example on account of a fault, such as incorrectly assembled parts or a forgotten seal during preparation, and/or on account of a functional error, such as a fatigue fracture.

DE 195 00 893 A1 discloses a device for crushing concretions, said device having a hollow probe, wherein the hollow probe consists of a tube, a probe base and a hollow-probe tip, and these three components can be plugged into one another and secured to one another via a soldered connection. The probe base has distal and proximal disc pairs on the outside, between which a sealing element designed as an O-ring is arranged. Since the probe base moves in the distal direction during operation, there is in principle a danger that, in the event of a failure of the seals, an overpressure from a pneumatic drive arrangement will enter the cavity of the probe and thus reach the patient.

Especially in the case of ballistic and/or pneumatic lithotripters with a hollow probe, there is the disadvantage of compressed air entering the patient on account of user errors, such as incorrect assembly of the parts and/or forgotten parts after preparation, or a failure of a component, and of the patient being unintentionally exposed to compressed air via the hollow probe. In addition to the active use of compressed air for moving the projectile and a compressed air volume present on account of the movement of the projectile, an overpressure posing a potential danger to the patient can also originate from a compressed-air reservoir or a spring device for repulsion of the projectile. In the case of a distal-side compressed-air reservoir, the latter is connected to the interior of the acceleration tube via a connection and a switching valve, in order to move the projectile back to the proximal-side abutment after it has impacted the distal-side abutment. Due to the temporally clocked compressed air surges and the impact hammer principle, there is an increased danger of component fatigue and of occurrence of leaks.

The object of the invention is to improve the prior art.

The object is achieved by a medical instrument for treating a body, wherein the medical instrument has a carrier unit having an outside to an environment and at least one first interior space within the carrier unit, and an overpressure can be applied to the interior space, wherein the medical instrument has a pressure relief device having at least one elastic sealing element, wherein the at least one elastic sealing element is arranged in a first recess in and/or on the outside of the carrier unit and the at least one interior space is connected to the first recess by means of at least one pressure relief channel, such that, when the overpressure is applied to the at least one interior space, the overpressure can be discharged into the environment by means of the first recess being freed by the at least one elastic sealing element.

Thus, a fault-tolerant medical instrument having a pressure relief device as safety device is made available in which, in the event of an overpressure, the latter can be deliberately discharged into the environment, which avoids subjecting a patient to pressure. Thus, in the event of too high an overpressure in the interior space, the overpressure is deliberately discharged into the environment by means of the pressure relief device, thereby preventing onward travel of the overpressure into a patient and, consequently, mechanical damage to and/or contamination of the body of the patient. Since the elastic sealing element is arranged in a first recess in and/or on the outside of the carrier unit and thus of a housing and this recess is connected to the interior space by means of a pressure relief channel, the elastic sealing element acts as both a pressure relief valve and a check valve, while at the same time preventing fluid and/or contamination from the environment from entering the pressure relief channel and the interior space. In the event of too high an overpressure, the elastic sealing element detaches from the recess and frees the latter for through-flow and for venting, as a result of which the at least one interior space connected via the pressure relief channel is relieved of pressure.

An essential concept of the invention is that at least one interior space in the carrier unit of a medical instrument, which is in fluid-communicating connection with the patient or in which a fluid-communicating connection can be established in the event of a fault, is connected via a pressure relief device to a pressure relief channel, which ends externally on and/or in the outside of the carrier unit in a recess in which at least one elastic sealing element is arranged, such that, in normal pressure operation, the elastic sealing element seals the carrier unit and thus the recess, the connected pressure relief channel and the interior space from the outside, but, in the event of too high an overpressure in the interior space, the elastic sealing element detaches from its sealing seat on and/or in the recess and thereby frees the recess, and therefore the overpressure can escape from the interior space via the pressure relief channel and the freed recess into the environment around the carrier unit. Thus, the pressure relief device constitutes a safety device, with which onward travel of an overpressure into the body of the patient is prevented during use of the medical instrument. The overpressure that triggers the venting overcomes in particular the weight, the inherent stress of the elastic sealing element and/or optionally, depending on the arrangement of the sealing element on and/or in the recess, the static friction forces between the sealing element and a surface of the recess.

It is particularly advantageous that the pressure relief device can be used for an overpressure generated by any media and/or fluids, and therefore an overpressure of a gas, of compressed air, of water, of a hydraulic fluid and/or of a heat transport means can be reduced and the corresponding interior space can be relieved of pressure.

In principle, it should be stressed that the object is also achieved by the carrier unit alone, without a complete medical instrument being formed. This is the case particularly when, for example in a lithotripter, the hollow probe is exchangeable and thus detachable from the carrier unit. Likewise, a shaft and/or a tool of the medical instrument can be detachable from the carrier unit and thus exchangeable, such that the carrier unit is also present on its own.

The following concepts should be explained:

A “medical instrument” is in particular any mechanical or mechanical/electrical unit of action which is suitable for the diagnosis and/or treatment of humans or animals. The medical instrument is used in particular for inspection of a human or animal body cavity and/or for manipulation of human or animal tissue. The medical instrument has in particular a carrier unit for holding and/or handling the instrument. Furthermore, the medical instrument can have a hollow probe, a shaft, a tool and/or an optical system for viewing a viewing region. The medical instrument can have in particular a gripping tool, a cutting tool, a needle holder, a clip applicator and/or another kind of tool. For example, a medical instrument can be an endoscope, with a long shaft and a bendable end section, or a lithotripter. The medical instrument can be a handheld and/or hand-guided instrument or a robot-assisted instrument. The medical instrument and/or the carrier unit can have, in particular, a feed device for supplying a pressure medium and/or fluid, such as compressed air, a hydraulic fluid and/or a heat transport means.

A “carrier unit” is in particular a handheld and/or holding part of the medical instrument and/or of a lithotripsy device. The carrier unit can be in particular a handle for manual and/or automated operation and/or a connection of the medical instrument. The carrier unit can also be arranged at, connected to and/or guided in an automated manner at a distal end of a robot arm. In particular, the carrier unit has a housing. Internally, the carrier unit has at least one first interior space, which can be subjected to an overpressure and/or in which an overpressure can arise. The “outside” of the carrier unit is in particular the side which is oriented toward the environment around the carrier unit and/or the medical instrument. Thus, the outside of the carrier unit is the side outside of a closed space of the carrier unit.

An “interior space” is in particular a space within the carrier unit protected from influences from the environment around the carrier unit and/or the medical instrument. In particular, the interior space is an enclosed space within the carrier unit.

An “overpressure” is in particular understood as a pressure measured relative to the atmospheric pressure and/or air pressure. An overpressure is in particular a pressure that is higher than the atmospheric pressure and/or air pressure. An overpressure is in particular a locally increased pressure within an interior space of the carrier unit. An overpressure within the interior space can occur in particular if, simultaneously, a larger volume flow of a fluid is introduced into the interior space than is removed. Likewise, an overpressure can arise if a space that is present is reduced without material being able to leave this space; for example, in the case of movement of a projectile in an acceleration tube of a lithotripter, the air volume is compressed in the direction of movement, such that an overpressure arises. For the overpressure, a setpoint can be specified from which, by means of the pressure relief device, the recess and/or of the sealing seat is freed by the at least one elastic sealing element and, therefore, the pressure applied to the interior space is discharged into the environment via the at least one pressure relief channel and the recess. In principle, the overpressure can occur through the supply of any kind of fluid into the interior space and/or by compression of this fluid.

A “pressure relief device” is understood in particular to mean any device by means of which a pressurized interior space and/or a region of the interior space can be relieved of pressure. By means of the pressure relief device, in particular an overpressure and/or an action of a fluid is prevented during use of the medical instrument and/or of the carrier unit in the body of a patient. The pressure relief device has in particular at least one elastic sealing element, which is arranged in a recess in and/or on the outside of the carrier unit, the recess being connected via at least one pressure relief channel to the pressurizable interior space of the carrier unit. Thus, the pressure relief device forms in particular a safety valve, pressure relief valve and/or check valve. By means of the pressure relief device, a fluid in the interior space is discharged, via the at least one pressure relief channel, an outlet opening of the pressure relief channel into the recess and through the recess when the outlet opening and/or the recess is freed by the at least one elastic sealing element, into the environment and thus into the atmosphere around the carrier unit and/or the medical instrument. This discharge of the fluid from the interior space into the environment by means of the pressure relief device takes place in particular when a predetermined demand pressure is exceeded. The pressure relief device is in particular a pressure relief device that is weight-loaded by means of the at least one elastic sealing element and/or configured with internal stress. In particular, the pressure relief device is free of any control. Thus, the pressure relief device is an automatic safety device against undesirable excess pressure.

A “sealing element” is in particular an element which prevents, limits or permits an unwanted transfer of material on both sides of the sealing element. A sealing element is in particular a static sealing element. The term “elastic” refers in particular to the property of the sealing element for changing its shape under the influence of force and for returning to the original shape when the acting force ceases to be applied. The elastic sealing element has in particular plastic and/or an elastomer as a dimensionally stable but elastically deformable plastic. The elastic sealing element can have, in particular, natural rubber and/or silicone rubber. In principle, the elastic sealing element can have any shape and can be formed, for example, as a elastic band, hose element, O-ring or X-ring. In terms of its form and/or its further properties, the elastic sealing element is designed in particular in such a way that it completely or at least partially occupies and/or fills the recess and thus seals the latter in normal operation of the medical instrument, but frees the recess in the critical case of an overpressure. The sealing element is inserted into and/or arranged in the recess in particular under force and compression, such that it is present in the recess under tension and seals the cavity of the recess at least partially or completely by virtue of its tensioned and flush sealing seat. When freed, the elastic sealing element detaches in particular from the transition between the pressure relief channel and the recess and/or from a sealing surface and/or sealing line in the recess.

A “recess” is in particular a recessed and thus free space in and/or on the outside of the carrier unit. The recess can be, in particular, an indentation, an incision, a notch, a hole, a gap, a groove, a depression and/or a molded cavity, open at the top, in the outer surface of the carrier unit.

A “pressure relief channel” is in particular a fluid connection by means of which an undesired overpressure can be led away from an interior space or a plurality of interior spaces to a recess or a plurality of recesses. The pressure relief channel can in principle have any shape, for example a channel that is square or round in cross section, a tube and/or a bore. In particular, the pressure relief channel ends with an outlet opening in the recess.

In a further embodiment of the medical instrument, the pressure relief device has a second elastic sealing element in a second recess in and/or on the outside of the carrier unit, a third elastic sealing element in a third recess in and/or on the outside of the carrier unit, and/or optionally a further elastic sealing element in a further recess in and/or on the outside of the carrier unit.

Thus, a single interior space can be relieved of pressure via at least one pressure relief channel and a plurality of locally distributed recesses, each with each an elastic sealing element.

A second, a third and/or optionally a further elastic sealing element corresponds in its function to the sealing element defined above, but the sealing elements can have different properties and/or shapes. Likewise, a second, a third and/or optionally a further recess corresponds to the recess defined above, and the recesses can also have different dimensions, shapes and/or further properties.

In order to make the pressure relief device safe and flexible and to relieve a plurality of interior spaces subjected to overpressure, a second interior space, a third interior space, a fourth interior space and/or optionally further interior spaces are arranged in the carrier unit, wherein the respective interior space can be subjected to an overpressure and can be connected via a respective pressure relief channel to the first recess, the second recess, the third recess and/or optionally the further recess.

Thus, a plurality of pressure relief channels from a plurality of different pressurized interior spaces can act on a single sealing element in a single recess. Thus, a large venting cross section is available on account of the multiple pressure relief channels routed to the single recess with an elastic sealing element. In addition, the design is simplified and more cost-effective and, on account of there being only one recess, installation space within the carrier unit is saved if multiple interior spaces are vented by one and the same elastic sealing element. Thus, the pressure relief device, with only a single elastic sealing element in a recess, serves as a safety valve for interior spaces of the carrier unit connected via multiple pressure relief channels. In addition to the cost-effective production of such a pressure relief device and the simple and rapid assembly during preparation, this also permits simple color coding and/or a color design. In addition, during maintenance, only a single sealing element in the single recess has to be checked for its functionality.

Likewise, the second and each further interior space, however, can also be separately connected via a pressure relief channel to an associated individual recess. This means that different fluids with in each case one overpressure can be diverted separately into the environment, and a backflow of the fluid into another pressure relief channel and/or interior space is prevented by the separate routing.

In a further embodiment of the medical instrument, the first recess, the second recess, the third recess and/or optionally the further recess is or are each connected to two or more pressure relief channels.

Thus, an overpressure from different regions of one interior space or from a plurality of different interior spaces can each be fed via two or more pressure relief channels into one recess or two or more recesses, respectively.

In order to ensure secure sealing in the normal operation of the medical instrument and reliable freeing of the recess in the critical case of an overpressure, the elastic sealing element, the respective elastic sealing element or the elastic sealing elements is/are formed as an elastic sealing band and/or elastic sealing ring.

Thus, the shape of the elastic sealing element can be selected specifically depending on the shape of the respective recess and/or vice versa. In the case of a rectangular recess and of an elastic sealing element with a rectangular cross section formed as a sealing band, a plurality of interior spaces can thus be controlled with the sealing band.

In a further embodiment of the medical instrument, the recess, the respective recess or the recesses is/are shaped conically on the side wall or side walls thereof.

This makes available a defined contact surface/sealing surface for the elastic sealing element within the recess, on which surface the sealing element can be arranged under tension. By virtue of the fact that the recess narrows conically for example in the direction of the pressure relief channel, the sealing element can be easily inserted and/or pressed into the recess. On the other hand, in the case of relief, the conical widening of the recess to the outside of the carrier unit facilitates and accelerates the detachment and lifting of the elastic sealing element from the sealing surface or sealing line on the respective side wall.

Preferably, a conically shaped recess with a sealing ring as an elastic sealing element is used only in connection with one interior space, since otherwise, upon connection to multiple interior spaces, there is a danger that these communicate and the excess pressure could be transferred from one interior space to another, without the excess pressure escaping to the outside. Therefore, in the case where the recess has conical side walls in which a sealing ring sits, a separate recess in and/or on the outside of the carrier element is preferably made available for each interior space and is in each case equipped with a separate sealing ring. Thus, a specially shaped recess with conical flanks and/or with a contact line for the sealing ring is made available.

In order in each case to specifically seal the region in the recess around the respective outlet opening of the pressure relief channel, the recess, the respective recess or the recesses can have a shaped contact surface for the respective elastic sealing element.

The “contact surface” is in particular a specifically shaped surface and/or a portion in and/or on the recess. The contact surface can also be a sealing seat, such that a defined sealing surface and/or sealing line is simultaneously provided by the shaped contact surface. The contact surface can be designed as a form-fitting counterpart to the sealing element and/or to a part of the sealing element. The contact surface can be, for example, a concentric ring around the outlet opening of the pressure relief channel, wherein the elastic sealing element, designed for example as a sealing ring, lies with its underside on the top of the ring and thus covers the outlet opening. Depending on the shape and/or size of the sealing surface and/or line and the weight and elasticity of the respective sealing element, it is possible to adjust the response pressure at which, in the case of a critical overpressure, the sealing element deforms and/or moves and consequently, by lifting off the contact surface, sealing surface and/or line, frees the outlet opening of the pressure relief channel and/or the sealing region of the recess. By means of a plurality of specially shaped contact surfaces in a single recess, two or more interior spaces in the same recess can be vented.

In a further embodiment of the medical instrument, the recess, the respective recess or the recesses has/have a sealing rib or two or more sealing ribs.

Thus, in the case of a plurality of interior spaces being connected to a single recess, an overflow of the fluid from one interior space to the other interior space can be prevented by sealing ribs surrounding each outlet opening of the pressure relief channel, since the sealing ribs each form circumferential separate sealing lines. The respective sealing ribs are arranged in such a way that they completely surround an outlet opening of the respective pressure relief channel and, with their top side, at the same time form a contact surface and thus sealing surface for the elastic sealing element.

In an embodiment in which a sealing band rests on a plurality of sealing ribs, the sealing ribs can be arranged in the central region of the recess in such a way that the outer region of the recess is free of sealing ribs, as a result of which a greater mobility is present at the outer edge of the sealing band and, in this way, the venting preferably takes place at the outer edge of the sealing band and not toward the center and/or in the direction of the other interior spaces.

A “sealing rib” is in particular a plate-shaped component and/or a plate. The sealing rib is arranged in particular vertically on the underside of the recess around and/or next to the outlet opening of the pressure relief channel into the recess. In particular, a plurality of sealing ribs can be arranged around the outlet opening. In particular, the sealing rib is cohesively bonded to the inner surface of the recess.

In order to permit heat removal from the medical instrument and/or removal of stone material, the medical instrument can have a fluid passage for transporting a crushed stone fragment and/or for heat transport.

In a further embodiment, the medical instrument is a ballistic lithotripsy device, in particular a ballistic intracorporeal lithotripsy device, for crushing stones in the body, wherein the lithotripsy device has a guide tube having a cavity, a proximal end and a distal end, a movable projectile, a distal-side abutment element and a proximal-side abutment element for the movable projectile, wherein the guide tube is arranged at least partially in the carrier unit, and the lithotripsy device can be assigned a drive device, for moving the projectile to and fro between the distal-side abutment element and the proximal-side abutment element, and a sonotrode, and the sonotrode, at its proximal end, is directly or indirectly connectable to the carrier unit and/or the guide tube and can be excited to oscillation by a mechanical impact of the projectile on the distal-side abutment element, wherein in the event of a fault the respective interior space can be subjected to the overpressure on account of a compression in the cavity of the guide tube caused by the reciprocating motion of the projectile.

Thus, a ballistic lithotripsy device is made available in which, in the event of a fault, an overpressure in the compressed air and/or gas volume in the cavity of the guide tube, caused by the reciprocating motion of the projectile, is deliberately discharged outward by means of the pressure relief device into the environment of the lithotripsy device and cannot enter the patient's body.

A “lithotripsy device” (also called a “lithotripter”) is in particular a device for crushing stones in the body by shocks, shock waves and/or deformation waves. A lithotripsy device is understood to include, in particular, various structural parts, constructional and/or functional components of a lithotripter The lithotripsy device can completely or partially form a lithotripter. A lithotripsy device can be in particular be an intracorporeal or extracorporeal lithotripsy device. In the case of an intracorporeal lithotripsy device, the latter can additionally have an irrigation/suction pump. The lithotripsy device can be designed as a handheld device and/or can have an endoscope or be inserted into an endoscope. The lithotripsy device is in particular autoclavable and has, for example, instrument steel and/or plastic. The lithotripsy device can have further components, such as a control and/or supply device, or these are assigned to the lithotripsy device.

In the “ballistic” lithotripsy device, a shock excitation of the sonotrode takes place as a result of reciprocating motion of the projectile and the distal-side impact of the projectile on the distal-side abutment element. Here, a ballistic movement occurs which is rapid and short-lasting and of which the course is not changeable or is only slightly changeable. In a “pneumatic” lithotripsy device, compressed air and/or a compressed gas is used for the driving and thus ballistic movement of the projectile.

The term “stones” (also called “concretions”) refers in particular to all stones in a human or animal body, which are formed for example from salts and proteins by crystallization and/or condensation. The stones can include gallstones, urinary stones, kidney stones and/or salivary stones.

A “projectile” is in particular a body which is freely movable within a cavity of a guide tube (also called acceleration tube) of a lithotripsy device along an acceleration section. The projectile is movable in particular in a reciprocating motion between a proximal-side abutment element and a distal-side abutment element within the cavity, arranged therebetween, of the guide tube. The projectile can also be surrounded by a control sleeve within the guide tube. In principle, the projectile can have any shape. For example, the projectile can have the shape of a bolt or a ball. The projectile has in particular hard steel and/or magnetic properties. For the free mobility, the projectile has in particular a slightly smaller outer diameter than the diameter of the cavity of the guide tube and/or of the control sleeve. For example, the projectile can have an outer diameter of 8 mm, preferably of 6 mm. The projectile can be moved in particular in a reciprocating motion between the proximal-side abutment element and the distal-side abutment element and thus along an acceleration path continuously, for example by means of a pressure medium of the drive device. Preferably, the projectile is moved to and fro in a continuously intermittent and/or oscillating manner between the proximal-side abutment element and the distal-side abutment element.

In principle, a “drive device” can be any type of device that places a force on the projectile and thus causes a movement of the projectile. The drive device can be, for example, a device which accelerates the projectile by means of a laser, a pressure medium, for example pneumatically by means of compressed air, by means of an electromagnetic field and/or by means of a mechanical device. In particular by supply and/or removal of a pressure medium, a drive device can place a force on the projectile and thus cause a movement of the projectile. In particular, the drive device permits a continuous and uniform flow of the pressure medium through the proximal and distal through-openings of the guide tube and proximal and distal openings of the control sleeve and an acceleration of the projectile within the cavity of the control sleeve and/or of the guide tube.

The terms “distal-side” and “distal” refer to an arrangement close to the patient's body and thus remote from the user, and/or a corresponding end or section. Accordingly, “proximal-side” or “proximal” refers to an arrangement close to the user and thus remote from the body, or a corresponding end or section.

A “sonotrode” (also called a “probe”) is in particular a component which, by the action and/or introduction of mechanical vibrations, is itself set in vibration, resonance vibration and/or deformation vibration. A sonotrode has in particular a headpiece (also called a “nipple”, “holding nipple” or “base body”) and an elongate insertion part, for example a probe tube or probe rod. The insertion part is in particular incorporated in a receiving unit in the thicker headpiece. The receiving unit is, for example, a bore in the headpiece, in which bore the proximal end and/or the proximal end section of the insertion part is joined firmly and/or non-releasably, for example soldered. In particular, a sonotrode is an elongate component. A sonotrode is, for example, at least partially rod-shaped, tubular and/or hose-shaped. The sonotrode can be a hollow probe. The sonotrode can be a one-piece or multi-piece sonotrode. The sonotrode has in the probe tube a diameter in a range of 0.5 mm to 4.5 mm, in particular of 0.8 mm to 3.8 mm. The sonotrode contains in particular steel, iron, cobalt, chromium, nickel, molybdenum, titanium, magnesium and/or aluminum alloys and/or carbon or glass composites. By means of the shock energy when the projectile strikes the distal-side abutment element and/or a proximal end of a spring device with intermediate storage and transfer of the kinetic energy, a specifically shaped deformation wave is impressed in particular on the sonotrode. The deformation wave causes in particular a translational movement of the distal end of the sonotrode, which causes improved crushing of stones on account of the large deflection. In addition to the mechanical shock, the sonotrode can additionally be excited to a vibration, in particular a longitudinal vibration, in particular by means of a vibration excitation device, for example with an ultrasonic vibration exciter. Thus, the sonotrode is designed in particular as a waveguide for the vibration waves generated by a vibration excitation device and/or for the shock waves and/or deformation waves of the projectile. The proximal end of the sonotrode can in particular bear directly or indirectly on the distal abutment element. Preferably, the headpiece of the sonotrode is mounted movably. The sonotrode is in particular shaped in such a way that it optimally introduces the vibration waves, deformation waves, shock waves and/or the ultrasonic vibration at its distal end into the body, into the region of the body to be treated, and/or directly onto the stone to be crushed in the body. It should be stressed here that, in the lithotripsy device in which the pressure relief device is used, it is usually not shock waves, but deformation waves that occur, since in this lithotripsy device there is no particle velocity faster than sound (in metals about 5,000 m/s).

In a further embodiment, the medical instrument is a pneumatic lithotripsy device and, by means of the drive device, a pressure medium can be fed into and/or removed from an interior of the carrier unit and/or of the guide tube for moving the projectile back and forth in the cavity of the guide tube, wherein in the event of a fault the respective interior space can be subjected to an overpressure by means of the pressure medium.

Thus, a pressure medium, such as compressed air and/or a compressed gas, is actively supplied, in the case of a pneumatic lithotripsy device, to the cavity of the guide tube for moving the projectile. There is therefore an increased risk that, due to an operating and/or functional error, an overpressure caused by the pressure medium arises, which can be safely discharged by means of the pressure relief device into the outer environment of the pneumatic lithotripsy device.

In a further embodiment, the sonotrode is a hollow probe.

In the case where the sonotrode is designed as a hollow probe and in the event of a fault, the pressure medium can in principle pass directly from the interior of the carrier unit through the hollow probe into the patient's body. This risk is prevented or at least significantly minimized by the pressure relief device according to the invention.

### BRIEF DESCRIPTION OF THE DRAWINGS

The invention is explained in more detail below on the basis of exemplary embodiments. In the drawing:

FIG. 1 shows a highly schematic three-dimensional view of a lithotripsy device,

FIG. 2 shows a highly schematic view of the lithotripsy device with a guide tube and a control sleeve in the longitudinal direction, during a movement of the projectile in the distal direction with a pressure relief valve,

FIG. 3 shows a highly schematic view of the distal region of the lithotripsy device from FIG. 2 with the pressure relief valve,

FIG. 4 shows a highly schematic view, in longitudinal section, of a pressure relief valve having a sealing ring in a conical recess of a carrier unit,

FIG. 5 shows, in longitudinal section, an alternative of the pressure relief valve with a sealing ring partially in a conical recess of a carrier unit,

FIG. 6 shows a highly schematic view, in longitudinal section, of the pressure relief valve from FIG. 3 with an elastomer band in a rectangular recess of the carrier unit,

FIG. 7 shows, in longitudinal section, a further alternative of the pressure relief valve with an elastomer band and sealing ribs in a rectangular recess of a carrier unit, and

FIG. 8 shows, in longitudinal section, a further alternative of the pressure relief valve with an elastomer band and sealing ribs in a rectangular recess, connected to two relief bores of a carrier unit.

### DETAILED DESCRIPTION

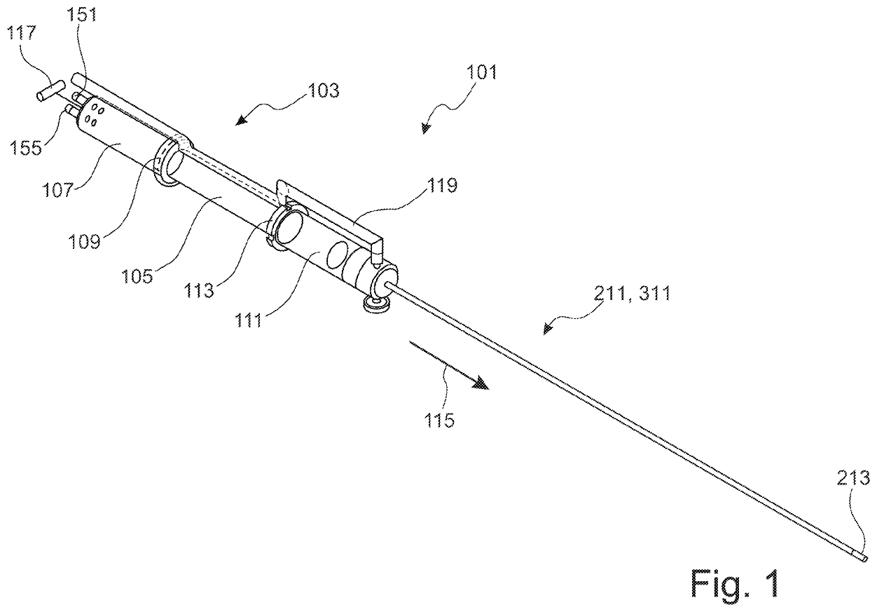

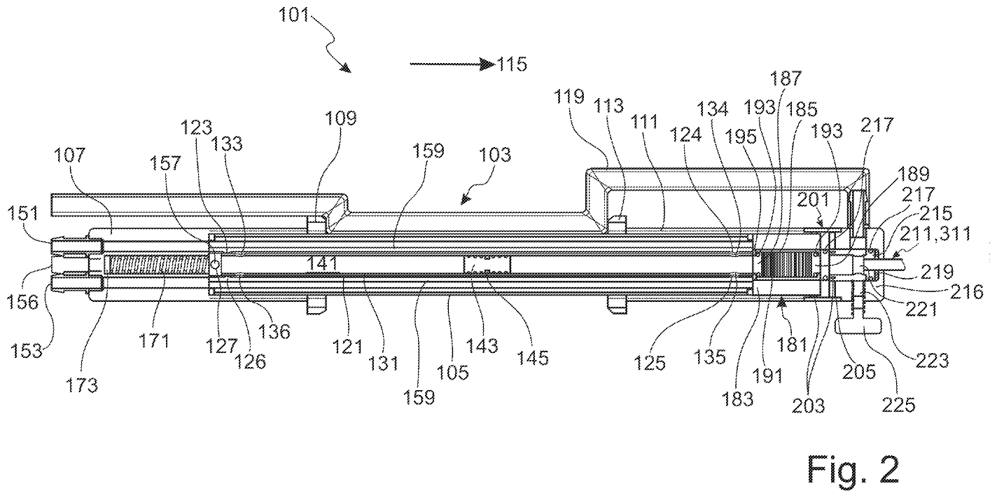

A lithotripsy device 101 has a carrier unit 103 having a central housing tube 105. At a proximal end of the housing tube 105, a proximal end cap 107 is screwed onto the housing tube 105 by means of a proximal lock nut 109. Likewise, at the distal end of the housing tube 105, a distal end cap 111 is screwed on by means of a distal lock nut 113 (see FIG. 1 and FIG. 2). A first exhaust-air connection 151 and a second exhaust-air connection 153 (the latter not visible in FIG. 1) are arranged at the proximal end of the proximal end cap 107. Furthermore, a first supply-air connection 155 and a second supply-air connection 107 (the latter not visible in FIG. 1) are arranged at the proximal side of the proximal end cap 107. From the distal end portion of the distal end cap 111, a suction line 119 is routed to the proximal end of the lithotripsy device 101 for suction of body stone fragments counter to a distal direction 115. The suction line 119 is shown only symbolically in FIG. 1 and has impracticable narrow bending radii. Likewise, in FIG. 1, an operating element 117 at the proximal end of the carrier unit 103 is shown only symbolically; the operating element 117 in an alternative embodiment is optimally arranged ergonomically on the housing tube 105. The operating element 117 is designed with a control sleeve 131, arranged inside the housing tube 105, for starting and switching off and for single and/or continuous firing by means of the ballistic lithotripsy device 101. An elongate sonotrode 211 designed as a hollow probe 311 and having a sonotrode tip 213 is arranged at the distal end of the carrier unit 103.

In the interior of the housing tube 105 of the carrier unit 103, a guide tube 121 is arranged spaced apart from the housing tube 105, wherein, between an inner wall of the housing tube 105 and an outer wall of the guide tube 121, two supply-air chambers 157 and two exhaust-air chambers 159 are arranged symmetrically over the cross section, which chambers are connected, via bores in the proximal end cap 107, to the crosswise-arranged first exhaust-air connection 151 and the second exhaust-air connection 153 and also the first supply-air connection 155 and the second supply-air connection 156 (in FIG. 2 the supply-air chamber 157 lies behind a fifth through-bore 127 and is only visible through the latter, while the second supply-air chamber lies in front of the viewing plane). The supply-air chambers 157 and the exhaust-air chambers 159 extend over the entire length of the guide tube 121. The two exhaust-air connections 151, 153 and the two supply-air connections 155, 156 are each connected via a Y-connector (not shown) to an exhaust-air hose and a supply-air hose of a drive device (not shown).

The guide tube 121 has, at the proximal side, a first through-bore 123 and a fourth through-bore 126, which are each connected to one of the two exhaust-air chambers 159 respectively. At the distal side, the guide tube 121 has a second through-bore 124 and a third through-bore 125, which are each connected to one of the two exhaust-air chambers 159 respectively. Moreover, at the proximal side, the guide tube 121 has the fifth through-bore 127 and a further through-bore lying opposite (and therefore not visible in FIG. 2), which are each connected to one of the two supply-air chambers 157 respectively (the two corresponding distal-side through-bores connected to the supply-air chambers 157 are not shown in FIG. 2). All of the through-bores 123, 124, 125, 126, 127 pass transversely through the lateral surface of the guide tube 121 and each have a diameter of 3 mm.

Arranged internally in the guide tube 121 is the control sleeve 131, which has a first valve bore 123 and a fourth valve bore 136 on the proximal side corresponding to the proximal-side through holes 133, 126 of the guide tube 121. Accordingly, a second valve bore 134 and a third valve bore 135 are introduced in the control sleeve 131 at the distal side. The control sleeve 131 is arranged in a manner protected against rotation within the guide tube 121, such that the corresponding through-bores of the guide tube 121 and the valve bores of the control sleeve 131 are freely continuous in a respective valve opening position. The control sleeve 131 has on the inside a cavity 141, which at the same time forms an acceleration path for a projectile 143. The projectile 143 has, on its outer surface, a driver ring 145, which bears externally on an inner surface of the control sleeve 131. The control sleeve 131 is 4 mm shorter than the guide tube 121.

At the proximal side of the control sleeve 131, a return spring 171 is arranged in a sheath tube, which is held in the proximal end cap 107 by means of a bracket 173.

At the distal end of the control sleeve 131, a prolongating spring 181 is arranged for imprinting a defined deformation wave on the sonotrode 211 on account of the mechanical impact of the projectile 143. The prolongating spring 181 has a plurality of stacked polymer disks 191 in the distal direction 115, which are surrounded externally by a sheath tube 185. The sheath tube 185 is held in the distal end cap 111 by means of a holder 183. At the proximal end of the sheath tube 185, a proximal closure cap 187 is arranged which has an O-ring 193 on the inside and is movably trapped and gripped by means of a welding ring 195, which is welded to the sheath tube 185. At the distal side, a distal closure cap 189 is arranged which is likewise movably gripped by means of a beading of the sheath tube 185 and also has an O-ring 193 on the inside.

Thus, the distal end of the return spring 171 is a proximal abutment element and the proximal closure cap 187 of the prolongating spring 181 is a distal abutment element for the projectile 143. FIG. 2 shows the state in which the control sleeve 131 is struck in a distal direction 115 against the distal-side abutment element formed by the proximal closure cap 187 of the prolongating spring 181. Since the control sleeve 131 is 4 mm shorter than the guide tube 121, the cavity of the guide tube 121 in the region of the proximal-side fifth through-opening 127 is free of the control sleeve 131. After repulsion of the projectile 143 on the closure cap 187 of the prolongating spring 181, the projectile 143 moves back counter to the distal direction 115 and takes the control sleeve 131 with it by means of the driver ring 145. By this reverse movement, the opposite distal-side through-openings (not shown in FIG. 2) of the guide tube 121 and the associated valve openings for the entry of supply air under pressure into the cavity 141 of the control sleeve 131 are freely continuous, and the incoming supply air pushes the projectile 143 further in the proximal direction. On account of this backward movement of the projectile 143 and the travel of the control sleeve 131, the first valve bore 133 of the control sleeve 131 is applied to the first through-bore 123 of the guide tube 121 and the fourth valve bore 136 of the control sleeve 131 to the fourth through-bore 126 of the guide tube 121 at a travel of 4 mm and upon a defined abutment of the proximal end of the control sleeve 131 against the distal-side end wall of the sheath tube of the return spring 171, as a result of which the respective through-bore and valve bore are continuous for the discharge of exhaust air into the exhaust-air chambers 159. At the same time, the fifth through-bore 127 and the opposite, non-visible further through-bore for the passage of supply air are closed. After repulsion of the projectile 143 at the proximal abutment by means of the return spring 171 and followed by a renewed movement in the distal direction 115, the control sleeve 131 is again entrained by means of the driver ring 145 away from the projectile 143 and, in this way, the fifth through-bore 127 and the opposite, non-visible further through-bore are opened. Supply air enters through the latter into the cavity 141 of the control sleeve 131 and moves the projectile 143 further in the distal direction 115, until the state shown in FIG. 2 is again reached.

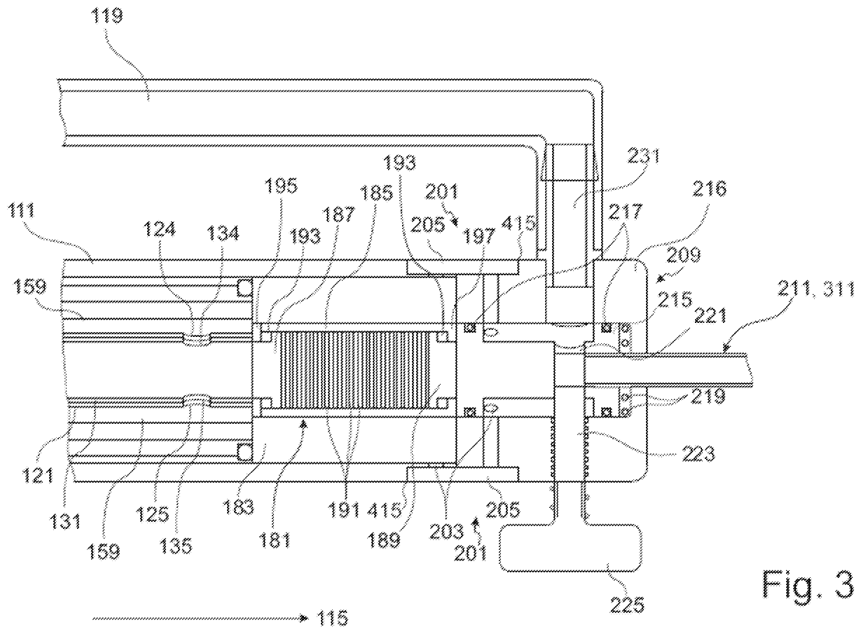

At the distal side of the prolongating spring 181, a head device 209 is arranged with a headpiece 215 of the sonotrode 211, wherein the headpiece 215, at its proximal end and its distal end respectively, is mounted movably in a guide part 216 by means of O-rings 217. The headpiece 215 has a transverse bore 221 in which an unblocking tappet 223, as a means protecting against rotation and for removing fragments of body stones, engages loosely with an actuation handle 225 (see FIGS. 2 and 3). The unblocking tappet 223 is held in position by means of a spring not shown in FIGS. 2 and 3. On the distal side of the head piece 215, a brake element 219 is arranged for limiting an amplitude of the sonotrode 211.

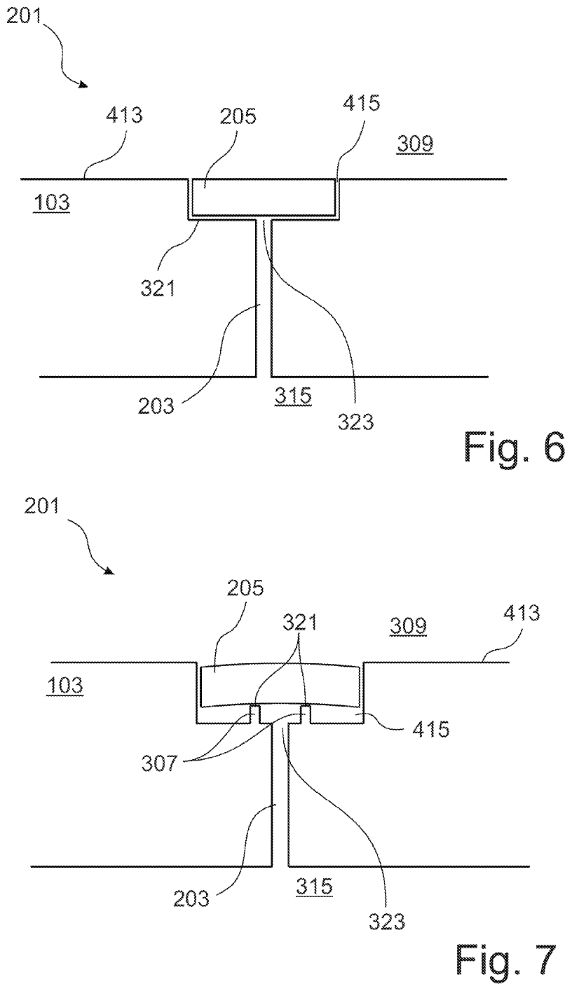

In the distal end cap 111, relief bores 203 are inserted to the headpiece 215 of the sonotrode 211 and to the holder 183 of the prolongating spring 181, which relief bores, together with a respective elastomer band 205 in a respective rectangular recess 415 in an outside of the carrier unit 103, respectively form a pressure relief valve 201 to the holder 183 of the prolongating spring 181 and to the headpiece 215 of the sonotrode 211 (FIG. 3), in order to prevent the effect of an overpressure in the patient in the event of a fault when using the lithotripsy device 101. One of the two pressure relief valves 201 is shown in a greatly simplified manner in FIG. 6. In the outer wall 413 of the carrier unit 103, oriented to the environment 309, the rectangular recess 415 is arranged in which the elastomer band 205 has been inserted under tension and thus has a circumferential sealing seat on the inner side walls of the rectangular recess 415. On its outside to the environment 309, the elastomer band 205 ends flush with the outer wall 413 of the carrier unit 103. At the underside of the elastomer band 205, the latter rests on a likewise sealing contact surface 321 of the rectangular recess 415. With its underside, the elastomer band 205 centrally seals an outlet opening 323 of the relief bore 203, which is connected to an interior 315 of the lithotripsy device 101.

A functional error in the operation of the lithotripsy device 101 with continuous supply of compressed air into the control sleeve 131 can occur, for example, as a result of fatigue or on account of an assembly error in the preparation of the proximal-side O-ring 193 of the sheath tube 215 of the prolongating spring 181 and of the O-rings 217 of the headpiece 215. The compressed air then passes in an undesired manner from the control sleeve 131 into the prolongating spring 181 and onward into the headpiece 215 of the sonotrode 211. As a result, there is a critical overpressure in the holder 183 of the prolongating spring 181 and in the headpiece 215. The critical compressed air flows from the holder 183 and headpiece 215 (interior 315 in each case) through a respective relief bore 203 and the respective outlet opening 323 into the respective rectangular recess 413, as a result of which the respective elastomer band 205 detaches from the rectangular recess 415 and is lifted, thereby causing the critical compressed air to escape into the environment 309. Through the two pressure relief valves 201, the critical compressed air in the prolongating spring 181 and in the headpiece 215 is discharged redundantly and safely into the environment 309 and cannot pass through the cavity of the sonotrode 211, formed as a hollow body 311, into the body of a patient under the action of the sonotrode tip 213 during the crushing of a body stone and potentially cause damage there to the body due to overpressure and/or contamination.

In an alternative of the pressure relief valve 201 as shown in FIG. 7, sealing ribs 321 are arranged circumferentially on the underside of the rectangular recess 321, on both sides of the outlet opening 323, each of them having, on their upper side, a contact surface 321 and thus a sealing seat with respect to the underside of the elastomer band 205. Otherwise, the pressure relief valve 201 is designed as described above with reference to FIG. 6. Since the elastomer band 205 rests with its sides on the side walls of the rectangular recess 415, but on its underside only rests on the contact surfaces 321 of the sealing ribs 307, then, in the event of a critical overpressure and therefore of compressed air flowing through the relief bore 203, the elastomer band 205 lifts rapidly away from the contact surfaces 231 and, on account of the free-hanging edges on both sides of the elastomer band 205, the latter is preferably lifted further in these edge regions, as a result of which the compressed air exits from the rectangular recess 415 into the environment 309.

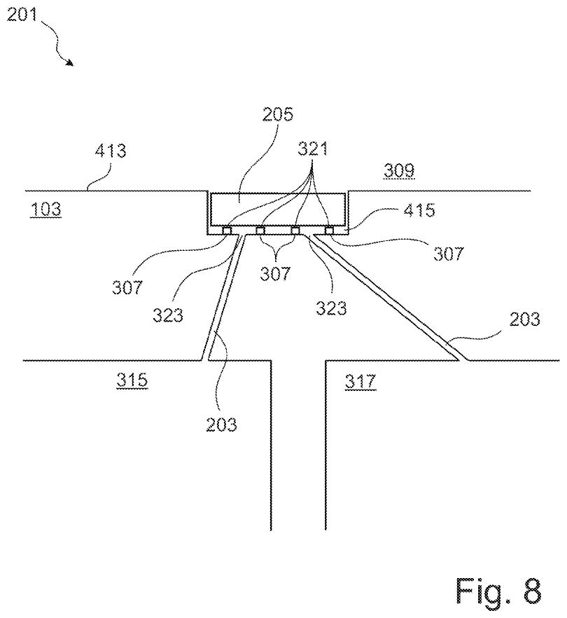

In another alternative of the pressure relief valve 201, the carrier unit 103 has a first interior space 315 and a second interior space 317 (FIG. 8). The two interior spaces 315 and 317 are each connected to a separate relief bore 203 with a single rectangular recess 415. The two outlet openings 323 of the two relief bores 203 are each surrounded by sealing ribs 307 within the rectangular recess 415, as a result of which two sealing ribs 307 are arranged centrally between the two outlet openings 323 and ensure a redundant seal between the two relief bores 203. As described above, the top sides of the sealing ribs 307 form the contact surfaces 321 and thus a sealing seat for the elastomer band 205. By virtue of the circumferential sealing ribs 307 in the rectangular recess 415, circumferential sealing lines are present in each case around the outlet opening 323, thereby preventing an overflow of compressed air from the first interior space 315 via the respective relief hole 203 into the second interior space 317, or vice versa. Otherwise, the alternative of the pressure relief valve 201, as shown in FIG. 8, is constructed as described above.

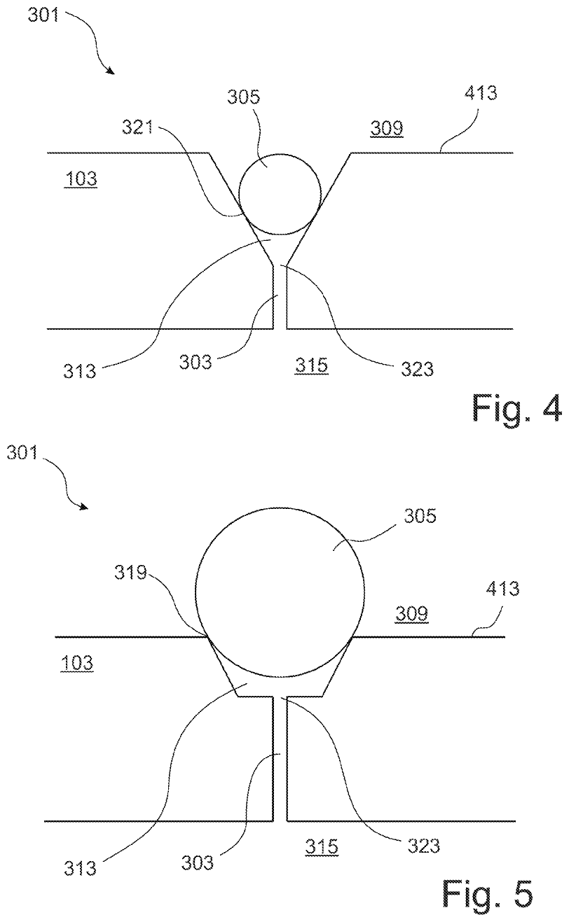

In a further alternative of the pressure relief valve, shown in FIG. 4, a pressure relief valve 301 has a conical recess 313, which conically narrows from the outer wall 413 of the carrier unit 103 to the outlet opening 323 and then transitions into a relief channel 303, which is connected at its other end to an interior 315 of the lithotripsy device 101. A sealing ring 305 has been pressed into the conical recess 313 and is clamped to a contact surface 321 of the side walls of the conical recess 313. In the event of a critical overpressure occurring, the compressed air flows from the interior space 315 through the relief channel 303 and the outlet opening 323 into the lower narrower region of the conical recess 313, which causes the sealing ring 305 to detach from the contact surface 321 and to rapidly lift off in the direction of the environment 309 due to the conical widening.

In a further alternative of the pressure relief valve 301, shown in FIG. 5, a sealing ring 305 is not completely received in a conical recess 313, but has a significantly higher height than the conical recess 313, as a result of which the sealing ring 305 largely projects into the environment 309. Due to the dimensions of the sealing ring 305, the underside of the latter does not lie on the underside of the conical recess 313 around the outlet opening 323, but forms a circumferential sealing line 319 at the edge of the conical recess 313 with respect to the outer wall 413. Thus, on account of the sealing ring 305, the pressure relief valve 301 is of a predominantly weight-loaded design and, in the event of a critical overpressure, the compressed air entering the conical recess 313 through the relief channel 303 and the outlet opening 323 must above all overcome the weight of the sealing ring 305 in order to detach the sealing ring 305 from the conical recess 313 at the sealing line 319.

Thus, in the lithotripsy device 101, pressure relief valves 201, 301 are provided with which interior spaces 315 can be vented safely and reliably and which can be unintentionally subjected to compressed air before the compressed air with a critical overpressure can reach the body of a patient via the hollow probe 311.

The invention relates to a medical instrument for treating a body, wherein the medical instrument has a carrier unit having an outside to an environment and at least one first interior space within the carrier unit, and an overpressure can be applied to the interior space, wherein the medical instrument has a pressure relief device having at least one elastic sealing element, wherein the at least one elastic sealing element is arranged in a first recess in and/or on the outside of the carrier unit and the at least one interior space is connected to the first recess by means of at least one pressure relief channel, such that, when the overpressure is applied to the at least one interior space, the overpressure can be discharged into the environment by means of the first recess being freed by the at least one elastic sealing element.

### LIST OF REFERENCE SIGNS

101 lithotripsy device
103 carrier unit
105 housing tube
107 proximal end cap
109 proximal lock nut
111 distal end cap
113 distal lock nut
115 distal direction
117 operating element
119 suction line
121 guide tube
123 first through-bore for exhaust air
124 second through-bore for exhaust air
125 third through-bore for exhaust air
126 fourth through-bore for exhaust air
127 fifth through-bore for supply air
131 control sleeve
133 first valve bore
134 second valve bore
135 third valve bore
136 fourth valve bore
141 cavity/acceleration section
143 projectile
145 driver ring
149 longitudinal center axis
151 first exhaust-air connection
153 second exhaust-air connection
155 first supply-air connection
156 second supply-air connection
157 supply-air chamber
159 exhaust-air chamber
171 return spring
173 bracket
181 prolongating spring
183 holder
185 sheath tube
187 proximal closure cap
189 distal closure cap
191 polymer discs
193 O-ring
195 welding ring
201 pressure relief valve
203 relief hole
205 elastomer band
211 sonotrode
213 sonotrode tip
215 headpiece
216 guide part
217 O-ring
219 brake element
221 transverse hole
223 tappet
225 actuation handle
301 pressure relief valve
303 relief channel
305 sealing ring
307 sealing rib
309 environment
311 hollow probe
313 conical recess
315 interior space
317 second interior space
319 sealing line
321 contact surface
323 outlet opening
413 outside wall
rectangular recess

1. A medical instrument configured to treat a body, wherein the medical instrument comprises:
a carrier unit having an outside to an environment and at least one first interior space within the carrier unit, and an overpressure can be applied to the interior space, wherein the medical instrument has a pressure relief device having at least one elastic sealing element, wherein the at least one elastic sealing element is arranged in a first recess in and/or on the outside of the carrier unit and the at least one interior space is connected to the first recess by at least one pressure relief channel, such that, when the overpressure is applied to the at least one interior space, the overpressure can be discharged into the environment by the first recess being freed by the at least one elastic sealing element.

2. The medical instrument according to claim 1, wherein the pressure relief device has a second elastic sealing element in a second recess in and/or on the outside of the carrier unit, a third elastic sealing element in a third recess in and/or on the outside of the carrier unit, and/or a further elastic sealing element in a further recess in and/or on the outside of the carrier unit.

3. The medical instrument according to claim 1, wherein a second interior space, a third interior space, a fourth interior space and/or further interior spaces is or are arranged in the carrier unit wherein the respective interior space can be subjected to an overpressure and is connected via a respective pressure relief channel to the first recess, the second recess, the third recess and/or the further recess.

4. The medical instrument according to claim 1, wherein the first recess, the second recess, the third recess and/or the further recess is or are connected to two or more pressure relief channels.

5. The medical instrument according to claim 1, wherein the elastic sealing element, the respective elastic sealing element or the elastic sealing elements is or are designed as an elastic sealing band and/or elastic sealing ring.

6. The medical instrument according to claim 1, wherein the recess, the respective recess or the recesses is or are shaped conically on the side wall or side walls thereof.

7. The medical instrument according to claim 1, wherein the recess, the respective recess or the recesses has or have a shaped contact surface element.

8. The medical instrument according to claim 1, wherein the recess, the respective recess or the recesses has or have a sealing rib or two or more sealing ribs.

9. The medical instrument according to claim 1, wherein the medical instrument has a fluid-carrying passage configured to transport a crushed stone fragment away and/or configured to transporting heat.

10. The medical instrument according to claim 1, wherein the medical instrument is a ballistic lithotripsy device configured to crush stones in the body, wherein the lithotripsy device comprises a guide tube having a cavity, a proximal end and a distal end, a movable projectile, a distal-side abutment element and a proximal-side abutment element for the movable projectile, wherein the guide tube is arranged at least partially in the carrier unit, and the lithotripsy device can be assigned a drive device, configured to move the projectile between the distal-side abutment element and the proximal-side abutment element, and a sonotrode, and the sonotrode, at its proximal end, is directly or indirectly connectable to the carrier unit and/or the guide tube and can be excited to oscillation by a mechanical impact of the projectile on the distal-side abutment element, wherein in the event of a fault the respective interior space can be subjected to the overpressure on account of a compression in the cavity of the guide tube caused by the reciprocating motion of the projectile.

11. The medical instrument according to claim 1, wherein the medical instrument is a pneumatic lithotripsy device and, by the drive device, a pressure medium can be fed into and/or removed from an interior of the carrier unit and/or of the guide tube for moving the projectile in the cavity of the guide tube, wherein in the event of a fault the respective interior space can be subjected to an overpressure by means of the pressure medium.

12. The medical instrument according to claim 1, wherein the sonotrode is a hollow probe.
